SEQUENCE LISTING (1 sequence)

SEQ ID NO: 1 - protein, 14 aa, Artificial Sequence
  GBP1
MHGKTQATSGTIQS

(19) United States
(12) Patent Application Publication
(10) Pub. No.: US 2014/0314668 A1
(43) Pub. Date: Oct. 23, 2014

(54) TECHNETIUM-99M LABELED COMPLEX OF GOLD NANOPARTICLE-GOLD BINDING PEPTIDES, AND METHOD OF MAKING AND USING THE SAME

(71) Applicant: Korea Atomic Energy Research Institute, Daejeon (KR)

(72) Inventors: Beom Su Jang, Daejeon (KR); Sang Hyun Park, Daejeon (KR); Joo-Sang Lee, Daegu (KR); Jong-Kook Rho, Jeongeup-si (KR)

(73) Assignee: Korea Atomic Energy Research Institute, Daejeon (KR)

(21) Appl. No.: 14/175,940

(22) Filed: Feb. 7, 2014

(30) Foreign Application Priority Data
Apr. 17, 2013 (KR) 10-2013-0042573

(51) Int. Cl. A61K 51/08

(52) U.S. Cl. CPC A61K 51/088
USPC 424/1.69; 530/327

(57) ABSTRACT

There is provided a method for preparing a ^{99m}Tc labeled gold nanoparticles-gold binding peptide, including: (a) coating gold nanoparticles with a gold binding peptide; and (b) labeling a composed ^{99m}Tc tricarbonyl precursor on the gold binding peptide coated on the gold nanoparticles. The ^{99m}Tc labeled gold nanoparticles-gold binding peptide prepared by the method according to the present invention is expected to be usefully employed for manufacturing a molecular contrast agent (imaging agent) which is traceable in organisms using imaging apparatuses such as gamma imaging and single photon emission computed tomography due to high labeling yield and good in-vivo stability.

### CROSS-REFERENCE TO RELATED APPLICATION

This application claims priority to and the benefit of Korean Patent Application No. 10-2013-0042573, filed Apr. 17, 2013, the disclosure of which is incorporated herein by reference in its entirety.

### BACKGROUND

1. Field of the Invention

The present invention relates to a method for preparing a technetium-99m (^{99m}Tc) labeled gold nanoparticles-gold binding peptide, and more specifically, a method for preparing a ^{99m}Tc labeled gold nanoparticles-gold binding peptide having high labeling yield and in-vivo stability, by easily binding a gold binding peptide to gold nanoparticles and attaching the gold binding peptide to a ^{99m}Tc tricarbonyl precursor, without introducing a chemical chelator to gold nanoparticles in gamma imaging/single photon emission computed tomography (SPECT) using ^{99m}Tc in the related art; a technetium-99m labeled gold nanoparticles-gold binding peptide prepared by the method; and use thereof.

2. Discussion of Related Art

Molecular imaging is a noninvasive technique for observing biological processes occurring in organisms at molecular and cellular levels and has been useful in the field of nuclear medicine. Particularly, positron emission tomography (PET) and single photon emission computed tomography technology has led to nuclear medical technology and performs many roles in clinical molecular imaging application.

X-ray computed tomography (CT) has been combined with PET and SPECT to develop a multi-modality imaging apparatus, which is innovative in the field of molecular imaging. PET/CT and SPECT/CT imaging systems, which are multi-modalities, can specifically target and provide the imaging of important cellular processes because both functional information and anatomic information can be provided by a single setting. The basis of molecular imaging in nuclear medicine is to use a molecular imaging agent in which includes radioisotopes such as ^{99m}Tc or ¹⁸F for visualizing and measuring biological processes in organisms. Particularly, ^{99m}Tc, a commercially available generator, is a good radioisotope in terms of its applicability and practicality because it enables diagnosis of gamma and SPECT imaging with a gamma camera and a SPECT scanner through a simple labeling method and separation process. Further, ^{99m}Tc is known to be an ideal radioisotope for performing diagnosis of tissue imaging due to its optimal γ-energy (i.e., 140 keV), suitable half-life (i.e., 6 hours), low cost, and wide availability.

Meanwhile, nanoparticles have been widely used for biomedicine and biotechnology, and particularly gold nanomaterials have drawn much attention due to their ease of synthesis and action, chemical stability, biocompatibility, and controllable optical and electrical properties. Accordingly, many studies involve manufacturing a multifunctional system that is biocompatible and stable for targeting a specific tissue, and functionalizing gold nanoparticles with a peptide or other organic molecules which may enable detection imaging of tumors, drug transfer and apoptosis using the multifunctional system (L. Sun, D. Liu, Z. Wang, Functional gold nanoparticle-peptide complexes as cell-targeting agents, Langmuir, 24, 2008, 10293-10297; N. Chanda, V. Kattumuri, R. Shukla, A. Zambre, K Katti, A. Upendran, R. R. Kulkarni, P. Kan, G. M. Fent, S. W. Casteel, C. J. Smith, E. Boote, J. D. Robertson, C. Cutler, J. R. Lever, K. V. Katti, R. Kalman, Bombesin functionalized gold nanoparticles show in vitro and in vivo cancer receptor specificity, Proc Natl Acad Sci USA, 107, 2010, 8760-8765; C. Kim, S. S. Agasti, Z. Zhu, L. Isaacs, V. M. Rotello, Recognition-mediated activation of therapeutic gold nanoparticles inside living cells, Nat Chem, 2, 2010 962-966; C. H. Choi, C. A. Alabi, P. Webster, M. E. Davis, Mechanism of active targeting in solid tumors with transferrin-containing gold nanoparticles, Proc Natl Acad Sci USA, 107, 2010, 1235-1240).

The related art using gamma imaging employing a radioisotope, ^{99m}Tc, which has been widely used, includes a method of attaching a chelator which may label ^{99m}Tc on the surface of nanoparticles. However, this method involves chemically introducing a chelator to gold nanoparticles and accordingly needs an additional step such as a purification process due to low labeling yield and in-vivo stability.

### SUMMARY OF THE INVENTION

The present invention is directed to providing a method for preparing a ^{99m}Tc labeled gold nanoparticles-gold binding peptide having high labeling yield and in-vivo stability, the method including: (a) coating gold nanoparticles with a gold binding peptide; and (b) labeling a ^{99m}Tc tricarbonyl precursor on the gold binding peptide coated on the gold nanoparticles.

In addition, the present invention is directed to providing a ^{99m}Tc labeled gold nanoparticles-gold binding peptide prepared by the method.

In addition, the present invention is directed to providing a molecular imaging agent composition and a nuclear medicine imaging contrast agent composition, including a ^{99m}Tc labeled gold nanoparticles-gold binding peptide.

However, technical problems solved by the present invention are not limited to those mentioned above, and other objectives, features and advantages of the invention may be clearly understood by those skilled in the art from the following description.

One aspect of the present invention provides a method for preparing a ^{99m}Tc labeled gold nanoparticles-gold binding peptide, including: (a) coating gold nanoparticles with a gold binding peptide; and (b) labeling a ^{99m}Tc tricarbonyl precursor on the gold binding peptide coated on the gold nanoparticles.

The gold binding peptide may be formed of an amino acid sequence of SEQ ID No. 1.

In addition, another aspect of the present invention provides a ^{99m}Tc labeled gold nanoparticles-gold binding peptide prepared by the method.

In addition, still another aspect of the present invention provides a molecular imaging agent composition including the ^{99m}Tc labeled gold nanoparticles-gold binding peptide.

In addition, yet another aspect of the present invention provides a nuclear medicine imaging contrast agent composition including the ^{99m}Tc labeled gold nanoparticles-gold binding peptide.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1A shows a result of color change according to aggregation of GNPs occurring in a variety of GBP1 concentrations;

FIG. 1B shows a UV-Vis absorbance spectral analysis result of FIG. 1A;

FIG. 2 shows radio-HPLC profiles of a ^{99m}Tc tricarbonyl precursor (a) and a ^{99m}Tc tricarbonyl labeled GBP (b);

FIG. 3 shows a schematic preparation process of a ^{99m}Tc labeled gold nanoparticles-gold binding peptide;

FIG. 4 shows radio-TLC profiles of a Na^{99m}TcO₄⁻ standard compound (a), a ^{99m}Tc(CO)₃ precursor standard compound (b), and a ^{99m}Tc labeled gold nanoparticles-gold binding peptide (c); and

FIG. 5 shows a distribution of a ^{99m}Tc labeled gold nanoparticles-gold binding peptide in an ICR mouse using micro-SPECT/CT imaging.

### DETAILED DESCRIPTION OF EXEMPLARY EMBODIMENTS

The present inventors studied a method of preparing an imaging agent having high in-vivo stability using a gold binding peptide, without introducing a chemical chelator to gold nanoparticles in gamma imaging/SPECT using ^{99m}Tc in the related art. As a result, they arrived at the present invention.

Hereinafter, the present invention will be described in detail.

The present invention provides a method for preparing a ^{99m}Tc labeled gold nanoparticles-gold binding peptide, including: (a) coating gold nanoparticles with a gold binding peptide; and (b) labeling a ^{99m}Tc tricarbonyl precursor on the gold binding peptide coated on the gold nanoparticles.

In other words, according to one Example of the present invention, a gold binding peptide (GBP) was added to gold nanoparticles above a minimum concentration to prevent aggregation of gold nanoparticles (GNP), followed by coating (see Example 1). A ^{99m}Tc tricarbonyl precursor was prepared (see Example 2) and labeled on the gold binding peptide coated on the gold nanoparticles to compose a ^{99m}Tc labeled gold nanoparticles-gold binding peptide, which had high radiochemical purity (see Example 3), and excreted through liver-biliary tract and kidney-urine pathways was confirmed using imaging (see Example 4).

In addition, the present invention provides a ^{99m}Tc labeled gold nanoparticles-gold binding peptide prepared by the method according to the present invention.

In addition, the present invention provides a molecular imaging agent composition including a ^{99m}Tc labeled gold nanoparticles-gold binding peptide according to the present invention.

In addition, the present invention provides a nuclear medicine imaging contrast agent composition including a ^{99m}Tc labeled gold nanoparticles-gold binding peptide according to the present invention.

Preferable methods of administering a molecular imaging agent composition or a nuclear medicine imaging contrast agent composition according to the present invention may include parenteral administration, such as bolus injection, intravenous injection or intra-arterial injection, spray, for example, aerosol spraying in the case of lung imaging, or oral or rectal administration, but are not limited thereto. Any administration method may be used so long as it is a known contrast agent administration method. The parenterally administrable form requires conditions free of germs, agents that are not physiologically acceptable, and any kind of paramagnetic, superparamagnetic, ferromagnetic, or ferrimagnetic contamination. The composition according to the present invention may be combined with preservatives, antimicrobials, buffer and antioxidants conventionally used for parenteral solution, excipients, and MR contrast agents, and may contain other additives without interrupting preparation, storage, or use of a product.

In addition, preferable dosage of a molecular imaging agent composition or a nuclear imaging contrast composition according to the present invention depends on patient condition and weight, degree of disease, drug type, administration route, and period, and may be suitably selected by those skilled in the art. The preferable dosage may be 1 to 1000 mg/kg of body weight at one time, and more preferably, 3 to 300 mg/kg of body weight at one time.

Hereinafter, preferable Examples will be described in order to facilitate better understanding of the present invention. However, the following Examples are not intended to limit the scope of the present invention.

### Preparation Example

### Preparation of Technetium-99m Labeled Gold Nanoparticles-Gold Binding Peptide According to the Present Invention

^{99m}Tc(CO)₃-GBP1-GNP according to the present invention was prepared using the following materials.

1) materials: 10 nM gold colloid solution (GNP-citrate) was manufactured by Sigma-Aldrich Korea (absorbance of 1.0 at 520 nm, 5.98×10¹² particles/ml) and a synthetic gold binding peptide 1 (GBP1, amino acid sequence (SEQ ID No. 1): MHGKTQATSGTIQS) was composed. Sodium pertechnetate (Na ^{99m}TcO₄⁻) was eluted from a ⁹⁹Mo/^{99m}Tc generator using 0.9% saline.

2) Experimental animals: Female ICR mice (7 weeks) were obtained from a specific pathogen-free colony at Orient, Inc. (Seoul, Korea) and used after quarantine and adaptation for one week. The animals were raised under 12 hours light/12 hours dark in a room maintained at a relative humidity of 50±5% and a temperature of 23±2° C., on a standard diet and water ad libitum. The animal experiment according to the present invention was carried out with the Institutional Animal Care and Use Committee's approval of Korea Atomic Energy Research Institute (KAERI).

3) HPLC apparatuses: An apparatus formed of agilent 1200 Series system (manufactured Agilent Technologies, Waldbronn, Germany) included a vacuum degasser, a double pump, a temperature control auto-sampler, a column oven separator, a UV-Vis detector, and a radioactive detector. HPLC was carried out using a Nucleosil C18 column (5 micron, 3.2×250 mm, Agilent Technology, Palo Alto, Calif., USA) at room temperature. A mobile phase was formed of methanol and 0.05 M TEAP buffer solution with gradient elution in an order of 100% methanol over 0 to 5 minutes; 0 to 25% 0.05 M TEAP buffer solution over 5 to 8 minutes; 25 to 34% methanol over 8 to 11 minutes; 34 to 100% 0.05 M TEAP buffer solution over 11 to 22 minutes; 100% 0.05 M TEAP buffer solution over 22 to 25 minutes. A flow rate was 0.6 ml/min and feed of the sample was 10 μL.

### Example 1

### Determination of Absorbance of GBP1 on the Surface of GNPs

In order to investigate a minimum concentration of GBP1 required for complete adsorption on the surface of gold nanoparticles (GNPs), since it is known that salts such as NaCl induce aggregation of gold and other nanoparitices (H.M.L. M. Y. Lin et al, Nature, 339, 1989, 360-362), an NaCl-induced aggregation method was used, and concentration of GBP1 adsorbed on the surface of GNPs was analyzed.

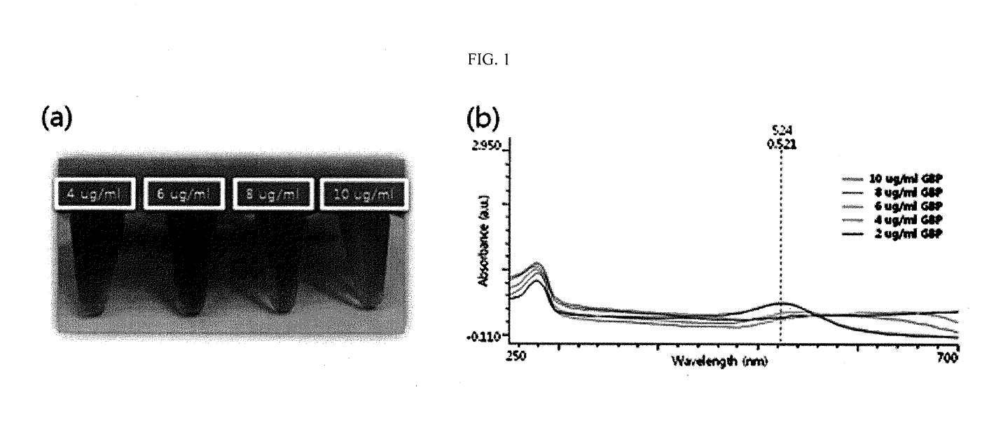

A variety of concentrations of GBP1 were added to 0.5 mL of GNP solution (O.D.=1.0, D=10 nm) to form a mixture, followed by standing for 10 minutes at room temperature. Next, 10% NaCl solution was added to the mixture so as to have a final concentration of 0.5% in order to induce aggregation of GNPs, followed by standing for 10 minutes at room temperature. Subsequently, in order to protect additional aggregation, 0.1% BSA was added to the resultant, followed by standing for 10 minutes at room temperature. Finally, aggregation degree of GNPs was measured in a wavelength range of 220 to 700 nm by UV-Vis spectrophotometer, and the measurement results are shown in FIG. 1.

Upon aggregation, an absorption spectrum was red-shifted so that color change from red to purple could be observed. As shown in FIG. 1A, concentration of GBP1 in GNP solution in the presence of NaCl was reduced from 10 μg/ml to 2 μg/ml, and it was confirmed that the GNP solution changed from red to purple. In addition, as shown in FIG. 1B, it was confirmed through UV-Vis absorbance spectral analysis that the main surface plasmon band of the GNP solution with GBP1 having a concentration of 10 μg/ml or less was red-shifted to 525 nm.

These results mean that aggregation of GNPs occurs at a low GBP1 concentration and is prevented at a GBP1 concentration of more than 10 μg/ml.

From the above, the present inventors used more than 10 μg/ml of GBP1 (20 μg/ml) to prepare GNPs coated with GBP1.

### Example 2

### Preliminary Experiment for the Preparation of ^{99m}Tc(CO)₃-Labeled GBP1

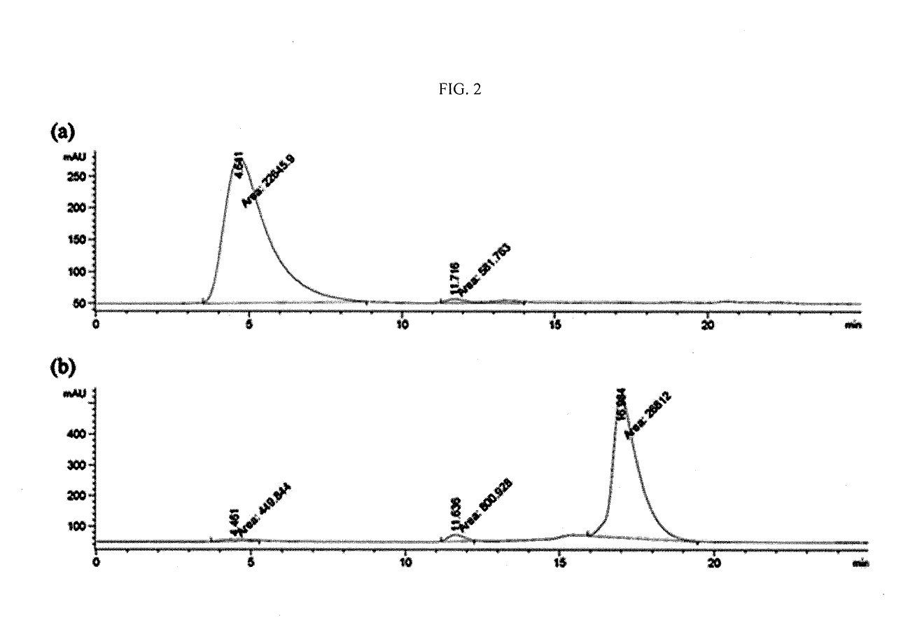

Before preparing ^{99m}Tc-GBP1-GNP, a preliminary experiment was carried out to find GBP1 to be labeled with a ^{99m}Tc(CO)₃ precursor. First, for radio-labeling, 5.9 mg of potassium boranocarbonate, K₂[BH₃CO]₂), 2.85 mg of sodium tetraborate, 8.5 mg of sodium tartrate, and 7.15 mg of sodium carbonate were added to a 5 mL bottle with a stopper, 1 ml of sodium pertechnetate (^{99m}TcO₄⁻, saline, 740 MBq) was added to the bottle using a syringe, and the bottle containing the mixture was heated for 30 minutes at 95° C. in a water bath to produce a ^{99m}Tc(CO)₃ precursor. Pressure from discharge gas (about 5 mL) was equalized by a 10 mL syringe. Subsequently, the resultant was cooled at room temperature and neutralized by adding 160 μL of 1 N HCl. The prepared ^{99m}Tc(CO)₃ precursor was reacted with GBP1 at pH 7 and heated for 2 hours at 75° C. Radioactive purity of ^{99m}Tc-GBP1 was determined through instant thin layer chromatography (ITLC) using a silica gel-impregnation fiber glass sheet (manufactured by Sciences, Pall Corp.). 5 μL of each sample was spotted on an ITLC strip, its radioactivity was measured using a TLC radioactive scanner (manufactured by Bioscan AR-2000, Washington D.C., USA), and the results are shown in FIG. 2.

According to the radioactive-HPLC analysis results shown in FIG. 2A, yield was 95% or more, residence time of the ^{99m}Tc(CO)₃ precursor was 4.6 minutes and residence time of the ^{99m}Tc(CO)₃ labeled GBP1 was 7.0 minutes. Further, as shown in FIG. 2B, it was confirmed from the average values of radioactive-HPLC, that the resultant had a radioactive purity of 95% or more and a yield of 95% or more.

These results mean that the ^{99m}Tc(CO)₃ precursor was present at a specific position (electron-donating group such as amine group or carboxyl OH group) of GBP1.

### Example 3

### Radioactive Aspect of ^{99m}Tc(CO)₃-Labeled, GNP Coated with GBP1

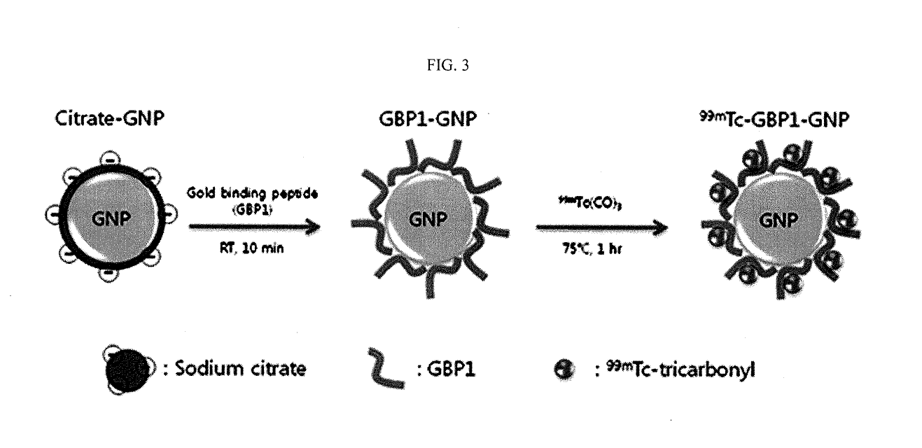

Based on the preliminary experiment results of Example 2, the GNPs coated with GBP1 were labeled with a ^{99m}Tc(CO)₃ precursor, and a preparation process of ^{99m}Tc-GBP1-GNPs is schematically shown in FIG. 3. In order to prepare GNPs coated with GBP1, 20 μL of GBP1 (1 mg/ml) was added to 1 mL of GNP solution (O.D.=1.0), followed by standing for 10 minutes at room temperature. Subsequently, the resultant was centrifuged for 30 minutes at 15,000 rpm, the supernatant (not bound to GBP1) was removed, and then pellets were resuspended with purified water so as to have a final absorbance of 1.0. 1 mL of GNPs coated with GBP1, having an absorbance of 1.0 in the purified water, was added to a protein LoBind tube (Eppendorf, Hamburg, Germany), 100 μL of a diluted ^{99m}Tc-tricarbonyl (^{99m}Tc(CO)₃) precursor (1 of 177.6 MBq (4.8 mCi)) was added thereto, the mixture was heated for 1 hour at 75° C. and centrifuged for 30 minutes at 15,000 rpm. Subsequently, the supernatant (^{99m}Tc-tricarbonyl precursor which had been not reacted) was removed, the removal process was performed twice, and pellets were redispersed in 300 μL of sterile saline (0.9% NaCl).

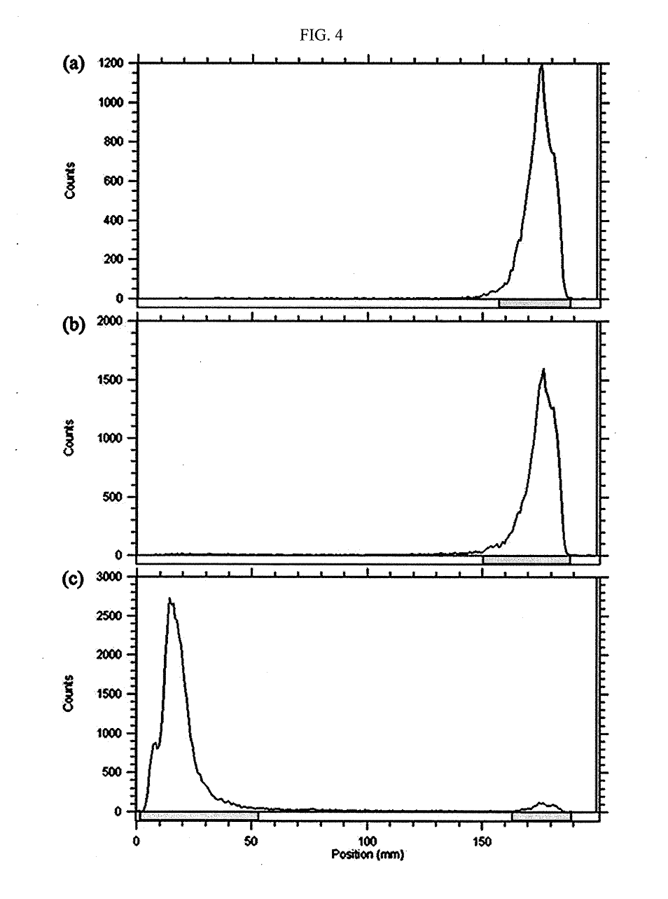

Further, radioactive purity of the labeled GNP was calculated from average values of radio-TLC using 0.1 M acetic acid as a developing solvent, and the results are shown in FIG. 4.

In the radioactive TLC, the labeled GNP was present at the origin, and a Na^{99m}TcO₄⁻ standard compound and a ^{99m}Tc(CO)₃ precursor standard compound moved with a solvent front as shown in FIGS. 4A and 4B (Rf=1.0). Further, radioactive TLC analyses of free ^{99m}Tc(CO)₃ and ^{99m}Tc(CO)₃ were compared with analysis of ^{99m}Tc(CO)₃ labeled GNP, and it was confirmed that high radioactive purity (>95%) was obtained, as shown in FIG. 4C.

### Example 4

### Imaging of Intravenously Administered ^{99m}Tc-GBP1-GNP in Organisms

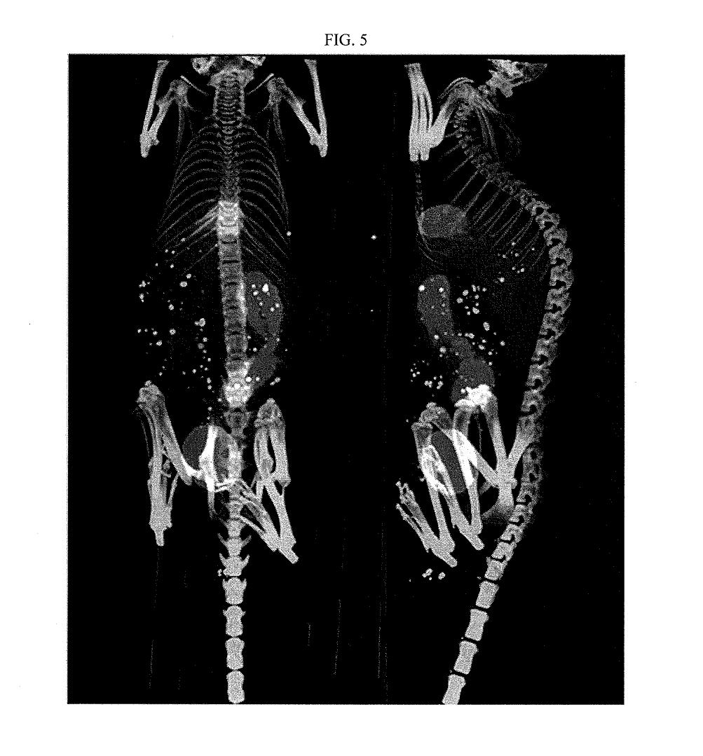

In order to analyze behavior of ^{99m}Tc-GBP1-GNP according to the present invention in organisms, the ^{99m}Tc-GBP1-GNP was intravenously administered to a mouse and imaged. A small animal Inveon SPECT/CT system (Siemens Medical Solutions) equipped with a 5-pinhole (mouse whole body) collimator was used for scanning the mouse. The mouse was anesthetized with 2% isofluran, while lying face down, micro-SPECT/CT imaging thereof was obtained by SPECT and CT scanning at 1 hour and 3 hours after 200 μL of ^{99m}Tc(CO)₃ labeled GNPs coated with GBP1 (18.5 MBq) was intravenously injected. At 1 hour after injection, distribution of the ICR mouse in organisms was measured by micro-SPECT/CT imaging, and the results are shown in FIG. 5.

As shown in FIG. 5, it was confirmed that Tc(CO)₃ labeled GNPs coated with GBP 1 was concentrated in a significant amount in the liver and on some level in the small intestine. The ^{99m}Tc(CO)₃ labeled GNPs coated with GBP1 was highly concentrated in the gallbladder and the urinary bladder and was negligible in other organ tissues.

These results mean that ^{99m}Tc(CO)₃-labeled, GNPs coated with GBP1 was excreted through liver.biliary tract and kidney pathways. ^{99m}Tc-GBP1-GNP was concentrated in the liver, which may be related to nano size and passive adsorption due to interaction with liver Kupffer cells.

Further, GNPs may easily aggregate in blood, saliva, or a biological culture medium because they were exposed to ions and proteins, but GNPs coated with GBP1 did not aggregate during the experiment. In addition, according to a result of micro-SPECT/CT imaging at 3 hours after the injection, radioactivity was not observed in the thyroid gland. Therefore, it was known that a tracer injected to the tail vein of the mouse was stable and did not decompose into free ^{99m}Tc pertechnetate form in organisms for at least 3 hours.

The ^{99m}Tc labeled gold nanoparticles-gold binding peptide prepared by the method according to the present invention can be usefully employed for manufacturing a molecular imaging agent composition that is traceable in organisms using imaging apparatuses such as gamma imaging and single photon emission tomography due to high labeling yield and good in-vivo stability.

The above description pertains to exemplary embodiments of the present invention, and those skilled in the art will understand that various changes in form and details can be made without changing the central technical ideas and essential features of the invention. Accordingly, the above description should not be construed as limiting the scope of the invention in any way.

What is claimed is:

1. A method of preparing a technetium-99m labeled complex of gold nanoparticle-gold binding peptides, the method comprising:
mixing gold nanoparticles with gold binding peptides to attach gold binding peptides to a surface of a gold nanoparticle; and
labeling with a technetium-99m tricarbonyl precursor at least part of the gold binding peptides attached to the gold nanoparticle.

2. The method of claim 1, wherein the gold binding peptides comprise an amino acid sequence of SEQ ID No. 1.

3. A technetium-99m labeled complex of gold nanoparticle-gold binding peptides, the complex comprising:
a gold nanoparticle;
gold binding peptides attached onto a surface of the gold nanoparticle; and
a technetium-99m labeling connected to at least part of the gold binding peptides.

4. A molecular imaging agent comprising the technetium-99m labeled complex of gold nanoparticle-gold binding peptides of claim 3.

5. A nuclear medicine imaging contrast agent comprising the technetium-99m labeled complex of gold nanoparticle-gold binding peptides of claim 3.

6. A method of molecular imaging, the method comprising:
administering the molecular imaging agent of claim 4 to a subject in need of such imaging; and
monitoring behavior of the agent in the subject using an imaging system.
